(19) United States
(12) Patent Application Publication
(10) Pub. No.: US 2012/0247500 A1
(43) Pub. Date: Oct. 4, 2012

(54) PROCESS FOR PERMANENTLY RESHAPING HAIR USING MERCAPTOSILOXANE COMPOSITION

(76) Inventors: Gregory Plos, Paris (FR); Patrice Lerda, Asnieres (FR); Anne Bouchara, Paris (FR)

(21) Appl. No.: 13/499,264

(22) PCT Filed: Sep. 23, 2010
(86) PCT No.: PCT/EP10/64031

§ 371 Date: Jun. 11, 2012

Related U.S. Application Data
(60) Provisional application No. 61/251,384, filed on Oct. 14, 2009.

(30) Foreign Application Priority Data
Sep. 29, 2009 (FR) 0956762

(51) Int. Cl. A45D 7/06; A61Q 5/04; A61K 8/58

(52) U.S. Cl. 132/206; 424/70.5

(57) ABSTRACT

A process for the cosmetic treatment of hair during an operation for permanently reshaping the hair, comprising a step of applying a composition comprising a silicone functionalized with one or more mercapto groups having a molecular weight of less than 10,000, and also to the use of this process and to a particular composition comprising the functionalized silicone.

The invention relates to a process for the cosmetic treatment of the hair during an operation for permanently reshaping the hair, comprising a step of applying a composition comprising a silicone functionalized with one or more mercapto groups having a molecular weight of less than 10 000, and also to the use of this process and to a particular composition comprising the functionalized silicone.

The first technique for obtaining permanent reshaping of the hair consists, in a first stage, in opening the —S—S— disulfide bonds of keratin (keratocystine) using a composition containing a suitable reducing agent such as ammonium thioglycolate and sulfites (reduction step), and then, after having rinsed the head of hair thus treated, generally with water, in reconstituting said disulfide bonds, in a second stage, by applying to the hair, which has been placed under tension beforehand, for example with rollers, an oxidizing composition (oxidation step, also known as the fixing step) so as to finally give the hair the desired shape. This technique thus makes it possible to make the hair wavy (permanent wave process) and/or to straighten (relax) the hair. The new shape given to the hair by a chemical treatment such as that above is eminently long-lasting and especially withstands the action of washing with water or shampoo, as opposed to simple standard temporary reshaping techniques, such as hairsetting.

The second technique for obtaining permanent reshaping of the hair consists in performing a “lanthionization” operation using a composition containing a base belonging to the hydroxide family. This leads to replacement of the disulfide bonds (—CH₂—S—S—CH₂—) with lanthionine bonds (—CH₂—S—CH₂—). This lanthionization operation involves two consecutive chemical reactions:
The first reaction consists of a beta-elimination on the cystine triggered by a hydroxide ion, resulting in the rupture of this bond and the formation of dehydroalanine, as is represented in the following reaction scheme.

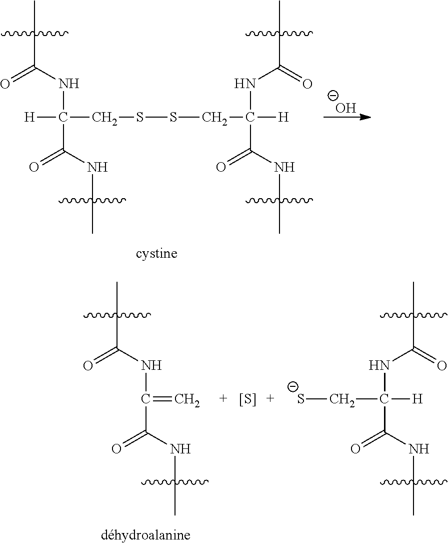

The second reaction is a reaction of the dehydroalanine with a thiol group. Specifically, the double bond of the dehydroalanine that is formed is a reactive double bond. It can react with the thiol group of the cysteine residue which was liberated in order to form a new bond known as a lanthionine bridge or bond or residue. This second reaction is illustrated by the following reaction scheme.

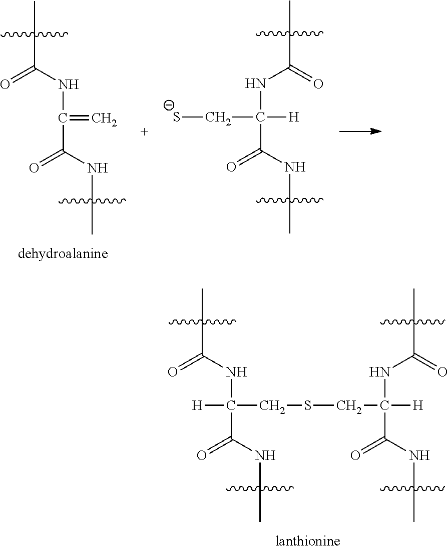

Compared to the first technique described above that uses a reducing agent, this lanthionization technique does not require a fixing step, since the formation of the lanthionine bridges is irreversible. It is therefore carried out in a single step and makes it possible either to make the hair wavy, or to shape it or uncurl it or straighten it out. However, it is mainly used for shaping naturally curly hair.

There is currently, in certain countries, and in particular in Brazil, a very strong consumer trend towards haircare treatments based on formol. These treatments provide volume control and control of humidity-mediated frizziness, and hair that is straight, well-managed and easy to style, and that feels repaired, and do so in a long-lasting manner.

Since the use of formol is prohibited owing to its toxic nature, there is a real need to look for nontoxic alternative solutions for obtaining the same effects.

The applicant has surprisingly discovered that by applying to the hair, during a permanent reshaping operation, such as a permanent wave, straightening, relaxing or uncurling operation, a composition comprising a silicone functionalized with one or more thiol groups and the molecular weight of which is less than 10 000, together with a step of applying heat, it was possible to give the hair a smooth feel, a decrease in weight and also volume control and control of humidity-mediated frizziness, these effects being persistent for at least 5 shampooing operations, and also an effect of relaxing curls on wavy and curly hair.

Document GB 1 182 939 describes a process for treating the hair, comprising a step of applying a reducing composition, a step of applying an aqueous dispersion of an organic silicone containing at least one mercaptoalkyl substituent, and then an oxidation step. This process makes it possible to make significant improvements in terms of sheen and body of the hair, and a shaping of the hair that is longer lasting in the presence of humidity.

Document U.S. Pat. No. 5,776,454 also describes a process for permanent shaping of the hair, giving said hair a conditioning effect. This process comprises the incorporation, into the reducing solution and/or into the oxidizing solution, of an emulsion comprising a thiolated silicone, prepared by emulsion polymerization.

Document EP 0 295 780 also describes a hair treatment process comprising the application of a reducing composition comprising an organic silicone functionalized with a mercaptoalkyl group, and then the application of an oxidizing composition. This process makes it possible to perform permanent shaping of the hair while at the same time conferring a conditioning effect resistant to washing.

However, none of the solutions proposed by these documents makes it possible to satisfactorily solve the problems indicated previously.

One subject of the invention is therefore a process for the cosmetic treatment of the hair, during an operation for permanently reshaping the hair, comprising:
a step of breaking the disulfide bonds of keratin, by applying, to the keratin fibers, a composition (A) comprising one or more agents for breaking disulfide bonds of keratin, then optionally
a fixing step aimed at reclosing said disulfide bonds, by applying an oxidizing composition (B) to the keratin fibers in the case of the application of one or more reducing agents as breaking agent,

it being understood that one or more silicones (i) having a molecular weight of less than 10 000 and that are functionalized with one or more mercapto groups are introduced into the composition (A) and/or into the oxidizing composition (B) and/or are applied to the keratin fibers between the step of applying the composition (A) and the step of fixing by applying the oxidizing composition (B), using an intermediate composition (C) containing it or them,

said process comprising a step of heating the hair to a temperature ranging from 60 to 220° C. after application of the silicone(s) (i).

Another subject of the invention is the use of the cosmetic treatment process to obtain a smooth feel of the hair, an increase in the lightness of the hair and a control of the volume of the hair in the presence of humidity.

The invention finally relates to kits that may be used for the implementation of the process according to the invention.

The invention finally relates to compositions comprising one or more silicones (i) having a molecular weight of less than 10 000 and that are functionalized with one or more particular mercapto groups.

The functionalized silicone(s) (i) used in the process according to the invention are chosen from compounds having the following formulae:

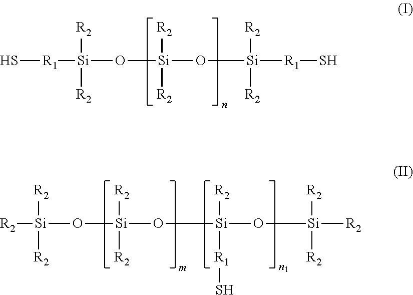

in which:

R₁ denotes a saturated or unsaturated, linear or branched, optionally cyclic hydrocarbon-based chain comprising from 1 to 100 carbon atoms, optionally interrupted with a heteroatom chosen from N, O, S and P,

R₁ preferably denotes a C₁-C₁₀₀ alkylene group, better still a propylene group,

R₂ denotes an alkyl group containing from 1 to 6 carbon atoms or an alkoxy group containing from 1 to 6 carbon atoms,

R₂ preferably denotes a methyl group or a methoxy group,

n ranges from 0 to 132,

n₁ ranges from 1 to 132, and

m ranges from 1 to 132.

As explained previously, the functionalized silicone(s) (i) are introduced into the composition (A) comprising one or more agents for breaking disulfide bonds of keratin, and/or into the oxidizing composition (B) and/or into an intermediate composition (C). Preferably, the functionalized silicone(s) are introduced into the composition (A).

The functionalized silicone(s) (i) may be introduced into the compositions of the invention, and in particular into the composition (A), just before the application of said compositions to the keratin fibers.

The functionalized silicone(s) (i) may be introduced into the composition (A) and/or (B) and/or (C) either in pure form or in the presence of one or more silicone-based or hydrocarbon-based solvents, or in the form of a latex.

The content of functionalized silicone (i) in composition (A) and/or (B) and/or (C) generally ranges from 0.5% to 50% by weight, preferably from 0.5% to 5% by weight and better still from 1% to 2% by weight relative to the total weight of the composition containing it.

As functionalized silicones that can be used in the process according to the invention, mention may be made of the mercaptosiloxane in which the mercapto functions are located at the chain end, sold by the company Shin-Etsu under the reference X-22-167B, and the mercaptosiloxane in which the mercapto functions are pendent, sold by the company Shin-Etsu under the reference KF-2001.

The composition (A) used for breaking the disulfide bonds of keratin in the process according to the invention generally comprises one or more agents for breaking disulfide bonds of keratin chosen from thiol reducing agents, non-thiol organic reducing agents, mineral and/or organic hydroxides and/or precursors thereof.

The thiol reducing agents are generally chosen from the reducing agents of formula

H—X—R

in which:

X represents a sulfur atom, and

R represents a linear, branched or cyclic, saturated or unsaturated C₁-C₂₀ hydrocarbon-based radical, or a formamidino or guanidine group, optionally interrupted with one or more heteroatoms, and optionally comprising one or more substituents chosen from one or more hydroxyl, carboxyl (COOH), (C₁-C₃₀)alkylcarboxyl (COOR), thiol, oxy (CO), amino, mono(C₁-C₃₀)alkylamino, di(C₁-C₃₀)alkylamino, amido (CONH₂), (C₁-C₃₀)alkyl-CON—, mono(C₁-C₃₀)alkyl-NCO— or di(C₁-C₃₀)alkyl-NCO— groups, or a halogenated group, the group(s) being linear, branched or cyclic, saturated or unsaturated C₁-C₂₀ hydrocarbon-based radical(s), optionally interrupted with one or more heteroatoms, and optionally comprising one or more substituents chosen from one or more hydroxyl, carboxyl (COOH), (C₁-C₃₀)alkylcarboxyl (COOR), thiol, oxy (CO), amino, mono(C₁-C₃₀)alkylamino, di(C₁-C₃₀)alkylamino, amido (CONH₂), mono(C₁-C₃₀)alkylamido or di(C₁-C₃₀)alkylamido groups, or a halogenated group,

when two alkyl substituents are borne by one and the same nitrogen atom, they may form a C₅-C₇ ring, such as a piperazine, which may be unsubstituted or substituted with one or more alkyl, hydroxyalkyl, amino or alkylamino groups, or a halogen atom, or a salt thereof in combination with a base.

By way of thiol reducing agents that can be used in the reducing composition used according to the invention, mention may be made of the thiol reducing agents chosen from thioglycolic acid, thiolactic acid, glycerol monothioglycolate, cysteamine, N-acetylcysteamine, N-propionylcysteamine, cysteine, N-acetylcysteine, thiomalic acid, pantetheine, 2,3-dimercaptosuccinic acid, N-(mercaptoalkyl)-ω-hydroxyalkylamides, N-mono- or N,N-dialkylmercapto-4-butyramides, aminomercaptoalkylamides, derivatives of N-(mercaptoalkyl)succinamic acids and of N-(mercaptoalkyl)succinimides, alkylaminomercaptoalkylamides, the azeotropic mixture of 2-hydroxypropyl thioglyconate and (2-hydroxy-1-methyl)ethyl thioglycolate, mercaptoalkylaminoamides, N-mercaptoalkylalkanediamides, and salts thereof.

The non-thiol organic reducing agents may be chosen from phosphines, sulfites, sulfinic compounds, borohydrides, reducing sugars and reductones.

As examples of phosphines, mention may be made of monophosphines or diphosphines as described in the FR2870119 series.

As examples of sulfinic compounds, mention may be made of derivatives of formamidine sulfinic acid.

As examples of borohydrides, mention may be made of sodium borohydride or potassium borohydride.

As examples of reducing sugars, mention may be made, in a nonlimiting way, of ribose, glucose, maltose, galactose, lactose or xylose.

As examples of reductones, mention may be made, in a nonlimiting way, of ascorbic acid and erythorbic acid.

For the purposes of the invention, the expression “mineral or organic hydroxides” means the compounds of structure (X^{p+})(OH⁻)_{q}

with X^{p+} denoting a mineral or organic cation, and

p and q being such that the electroneutrality of the compound is respected.

The mineral or organic hydroxides may be chosen from sodium hydroxide, lithium hydroxide, potassium hydroxide, calcium hydroxide and guanidine hydroxide.

As precursors of organic hydroxides, mention may be made only of guanidine carbonate.

The agent(s) for breaking disulfide bonds of keratin generally represent from 0.1% to 50%, preferably from 2% to 10%, by weight relative to the total weight of the composition (A).

The composition (A) used in the process according to the invention may comprise one or more cosmetic active agents.

This or these cosmetic active agent(s) are generally chosen from anionic, cationic, nonionic, amphoteric or zwitterionic surfactants, swelling agents and penetrants, disulfide compounds of reducing agents, for example dithioglycolic acid, thickening polymers of natural or synthetic origin, fixing polymers, conditioning agents such as cationic polymers, cations, cationic surfactants, silicones other than those of the invention and chitosans and derivatives thereof, hydrophobic solvents, fatty alcohols, direct dyes, in particular cationic or natural dyes, oxidation dyes, organic or mineral pigments, UV-screening agents, fillers, for example nacres, TiO₂, resins and clays, fragrances, peptizers, vitamins, amino acids, preserving agents and chelating agents.

The surfactants optionally present in the composition (A) used according to the invention are especially the following:

(i) Anionic Surfactant(s):

As examples of anionic surfactants that can be used, alone or as mixtures, within the context of the present invention, mention may especially be made (nonlimiting list) of the salts (in particular alkali metal, especially sodium, salts, ammonium salts, amine salts, amino alcohol salts or magnesium salts) of the following compounds: alkyl sulfates, alkyl ether sulfates, such as sodium lauryl ether sulfate, alkylamido ether sulfates, alkylaryl polyether sulfates, monoglyceride sulfates; alkyl sulfonates, alkyl phosphates, alkylamide sulfonates, alkylaryl sulfonates, α-olefin sulfonates, paraffin sulfonates; (C₆-C₂₄)alkyl sulfosuccinates, (C₆-C₂₄)alkyl ether sulfosuccinates, (C₆-C₂₄)alkylamide sulfosuccinates; (C₆-C₂₄)alkyl sulfoacetates; (C₆-C₂₄)acyl sarcosinates and (C₆-C₂₄)acyl glutamates. It is also possible to use (C₆-C₂₄)alkyl polyglycoside carboxylic esters such as alkyl glucoside citrates, alkyl polyglycoside tartrates and alkyl polyglycoside sulfosuccinates, alkyl sulfosuccinamates; acyl isethionates and N-acyl taurates, the alkyl or acyl radical of all of these various compounds preferably containing from 12 to 20 carbon atoms and the aryl radical preferably denoting a phenyl or benzyl group. Among the anionic surfactants that may also be used, mention may also be made of fatty acid salts such as oleic, ricinoleic, palmitic and stearic acid salts, coconut oil or hydrogenated coconut oil acids; acyl lactylates in which the acyl radical contains 8 to 20 carbon atoms. It is also possible to use alkyl D-galactoside uronic acids and salts thereof, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and salts thereof, in particular those containing from 2 to 50 alkylene oxide and in particular ethylene oxide groups, and mixtures thereof.

(ii) Nonionic Surfactant(s):

Nonionic surfactants are themselves also compounds that are well known per se (see especially in this regard the “Handbook of Surfactants” by M. R. Porter, published by Blackie & Son (Glasgow and London), 1991, pp. 116-178). Thus, they may be chosen in particular from (nonlimiting list) alcohols, alpha-diols, and alkylphenols, these compounds being polyethoxylated or polypropoxylated, and having at least one fatty chain containing, for example, from 8 to 18 carbon atoms, it being possible for the number of ethylene oxide and/or propylene oxide groups to range in particular from 2 to 50. Mention may also be made of ethylene oxide and propylene oxide copolymers, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; ethoxylated fatty acid esters of sorbitan containing from 2 to 30 mol of ethylene oxide; fatty acid esters of sucrose, fatty acid esters of polyethylene glycol, alkyl polyglycosides, N-alkyl glucamine derivatives, amine oxides such as (C₁₀-C₁₄)alkylamine oxides or N-acylaminopropylmorpholine oxides.

(iii) Amphoteric or Zwitterionic Surfactant(s):

The amphoteric or zwitterionic surfactants may especially be (nonlimiting list) aliphatic secondary or tertiary amine derivatives in which the aliphatic radical is a linear or branched chain containing from 8 to 18 carbon atoms and containing at least one water-solubilizing anionic group (for example carboxylate, sulfonate, sulfate, phosphate or phosphonate); mention may also be made of (C₈-C₂₀)alkyl betaines, such as cocobetaine, sulfobetaines, (C₈-C₂₀)alkylamido(C₁-C₆)alkyl betaines or (C₈-C₂₀)alkylamido(C₁-C₆)alkyl sulfobetaines.

Among the amine derivatives, mention may be made of the products sold under the name Miranol, as described in U.S. Pat. No. 2,528,378 and U.S. Pat. No. 2,781,354 and classified in the CTFA dictionary, 3rd edition, 1982, under the names Amphocarboxyglycinates and Amphocarboxypropionates, having the respective structures:

R₂—CONHCH₂CH₂—N(R₃)(R₄)(CH₂COO—)

in which: R₂ denotes a linear or branched C₅-C₂₀ alkyl radical originating, for example, from an acid R₂—COOH present in hydrolyzed coconut oil, a heptyl, nonyl or undecyl radical, R₃ denotes a β-hydroxyethyl group and R₄ a carboxymethyl group;

and

R₂′-CONHCH₂CH₂—N(B)(C)

in which:

B represents —CH₂CH₂OX′, C represents —(CH₂)_{z}—Y′, with z=1 or 2,

X′ denotes the —CH₂CH₂—COOH group or a hydrogen atom,

Y′ denotes —COOH or the —CH₂—CHOH—SO₃H radical,

R₂′ denotes a linear or branched, saturated or unsaturated, C₅-C₂₀ alkyl radical of an acid R₉—COOH present, for example, in coconut oil or in hydrolyzed linseed oil, an alkyl radical, especially a C₇, C₉, C₁₁ or C₁₃ alkyl radical, a C₁₇ alkyl radical and its iso form, or an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

An example that may be mentioned is the cocoamphodiacetate, sold under the trade name Miranol® C2M Concentrate by the company Rhodia Chimie.

(iv) Cationic Surfactant(s):

Mention may in particular be made (nonlimiting list) of: optionally polyoxyalkylenated, primary, secondary or tertiary fatty amine salts; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic nature.

Chelating agents that may be mentioned include diethylenetriaminepentaacetic acid and salts thereof.

The thickener(s) optionally present in the composition (A) used according to the invention may be chosen from cellulose-based thickeners, for example hydroxyethyl cellulose, hydroxypropyl cellulose and carboxymethyl cellulose, guar gum and derivatives thereof, for example the hydroxypropyl guar sold by the company Rhodia under the reference Jaguar HP 105, gums of microbial origin, such as xanthan gum and scleroglucan gum, synthetic thickeners such as crosslinked acrylic acid or acrylamido-propanesulfonic acid homopolymers, for example Carbomer, nonionic, anionic, cationic or amphoteric associative polymers, such as the polymers sold under the names Pemulen TR1 or TR2 by the company Goodrich, Salcare SC90 by the company Ciba, Aculyn 22, 28, 33, 44 or 46 by the company Röhm & Haas, and Elfacos T210 and T212 by the company Akzo. Mention may also be made of thickening polymers of polysaccharide type.

The pH of the composition (A) preferably varies between 7 and 14.

Preferably, the pH varies from 8 to 9.5, when the composition (A) comprises a thiol reducing agent.

Preferably, the pH varies from 11 to 14, when the composition (A) comprises an alkaline agent of hydroxide type.

The composition (A) may be in liquid form or in thickened form. It may be applied from a squeezy bottle, by brush or directly from the tube.

Generally, the composition (A) is left on for 5 minutes to 1 hour and preferably for 10 minutes to 30 minutes. After the optional leave-on time, the hair may be rinsed, preferably with water.

The oxidizing composition (B) used in the process according to the invention generally comprises one or more oxidizing agents chosen from hydrogen peroxide, alkali metal bromates, polythionates, persalts, such as perborates, percarbonates and persulfates, adsorbed or non-adsorbed metal salts, and enzymes of the 2-electron oxidase family.

Preferably, the oxidizing agent is hydrogen peroxide in the form of aqueous hydrogen peroxide solution, or alkali metal bromates.

Even more preferably, the oxidizing agent is hydrogen peroxide in the form of aqueous hydrogen peroxide solution.

The oxidizing agent(s) generally represents from 0.1% to 8% by weight and preferably from 0.2% to 5% by weight relative to the total weight of the oxidizing composition (B).

Preferably, when the oxidizing agent is hydrogen peroxide in aqueous solution, the oxidizing composition used in the process according to the invention contains at least one stabilizer of the aqueous hydrogen peroxide solution.

Mention may be made in particular of alkali metal or alkaline-earth metal pyrophosphates, such as tetrasodium pyrophosphate, alkali metal or alkaline-earth metal stannates, phenacetin or acid salts of oxyquinoline, for instance oxyquinoline sulfate (8-hydroxyquinoline sulfate). Even more advantageously, use is made of at least one stannate optionally in combination with at least one pyrophosphate.

The aqueous hydrogen peroxide solution stabilizer(s) generally represent from 0.0001% to 5% by weight and preferably from 0.01% to 2% by weight relative to the total weight of the oxidizing composition (B).

The oxidizing composition (B) used in the process according to the invention may also comprise one or more cosmetic active agents, such as those mentioned above with regard to the composition (A).

The oxidizing composition may be in liquid form or in thickened form.

Generally, the pH of the oxidizing composition ranges from 1 to 13 and preferably from 1.5 to 8.

When the oxidizing agent is hydrogen peroxide, the pH of the oxidizing composition (B) preferably varies from 1.5 to 5.

Generally, the oxidizing composition (B) is left on for 5 minutes to 1 hour and preferably for 10 minutes to 30 minutes.

The cosmetically acceptable medium of compositions (A) and (B), and optionally (C) used in the process according to the invention is preferably an aqueous medium formed by water and may advantageously contain one or more cosmetically acceptable organic solvents, more particularly including alcohols such as ethyl alcohol, isopropyl alcohol, benzyl alcohol and phenylethyl alcohol, or polyols or polyol ethers, for instance glycerol, ethylene glycol monomethyl, monoethyl and monobutyl ethers, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether. The organic solvents may then be present in concentrations of between about 0.1% and 20% and preferably between about 1% and 10% by weight relative to the total weight of the composition.

Generally, the composition is in the form of a solution, a dispersion or an emulsion. In the case where it contains the silicone according to the invention, the reducing composition is in the form of a dispersion. It is then shaken before use. The reducing composition may also be in the form of an emulsion that is sufficiently homogeneous to be applied directly to the hair.

The pH of compositions (A), (B) and (C) used in the process according to the invention may be obtained and/or adjusted conventionally by addition either of alkaline agents, for instance aqueous ammonia, monoethanolamine, diethanolamine, triethanolamine, isopropanolamine, 1,3-propanediamine, an alkali metal carbonate or bicarbonate or ammonium carbonate or bicarbonate, it being possible of course for all these compounds to be taken alone or as a mixture, or of acidifying agents such as, for example, hydrochloric acid, acetic acid, lactic acid, boric acid, citric acid and phosphoric acid.

As explained previously, the process according to the invention comprises a step of heating the hair to a temperature ranging from 60° C. to 220° C., after application of the silicone(s) (i).

If the silicone(s) (i) are present only in composition (A), the heating step is carried out after the application of compositions (A) or (B). This heating step may also be carried out after the application of an intermediate composition if this does not contain silicones (i).

If the silicone(s) (i) are present in composition (C), and optionally in composition (A), the heating step is carried out after the application of compositions (C) or (B).

If the silicone(s) (i) are present in composition (B), and optionally in compositions (A) and/or (C), the heating step is carried out after the application of composition (B).

This heating step is necessary for intensifying the particular effect of the functionalized silicone(s).

The step of heating the hair may be performed by drying the hair and/or by treating it with a heating appliance. In particular, the heating step may be performed using a hairdryer, a hood, heating rollers, a straightening iron, a curling iron, a crimping iron, a steam iron, an infrared lamp or an exothermic system in the composition. Preferably, the heating step is performed using a straightening iron.

According to one preferred embodiment, the step of heating the hair is performed at a temperature ranging from 80 to 220° C., preferably from 100 to 220° C. and better still from 100 to 180° C.

Generally, the process according to the invention comprises, after the oxidation fixing step, and after an optional leave-on time, a step of rinsing the hair, preferably with water.

One particular process according to the invention consists in applying the formulation containing at least one mercaptosiloxane having a weight of less than 10 000 several times, optionally interspersing one or more cosmetic treatments, in particular a shampooing operation, until the desired treatment intensity is obtained.

When it is desired to carry out a permanent-wave operation, mechanical means for placing the hair under tension, such as rollers, are preferably used.

In the context of a permanent-wave process, the steps below are followed.

Preferably, the composition (A) containing one or more reducing agents as defined above is applied to dry or wet, optionally shampooed, hair rolled beforehand onto rollers having a diameter of from 2 to 30 mm. The composition may also be applied gradually as the hair is rolled up.

Generally, the reducing composition is then left to act for a period of 5 minutes to 1 hour, preferably for 5 to 30 minutes. Rinsing can then be carried out.

Optionally, an intermediate rinse-off or leave-on composition (C) is then applied.

An oxidizing composition (B) for reforming the disulfide bonds of keratin is then applied to the rolled-up or unrolled hair, generally for a leave-on time of 1 minute to one hour. The head of hair is generally thoroughly rinsed, preferably with water. Final shampooing can be carried out.

The step of heating the hair to a temperature ranging from 60° C. to 220° C. is performed as indicated previously.

In the context of a hair relaxing or uncurling process, the steps below are followed, using either a reducing composition (A) or a composition comprising an alkaline agent.

When it is desired to relax or uncurl the hair, the composition (A) containing one or more reducing agents or one or more mineral or organic hydroxides or precursors thereof is applied to dry or wet hair.

The composition (A) is optionally left on for a period of 5 minutes to 1 hour, preferably for 5 to 30 minutes, and then the hair is rinsed in order to remove the composition (A).

The hair is straightened or uncurled generally using a comb. It can then be rinsed.

Optionally, an intermediate rinse-off or leave-on composition (C) is then applied.

Then, when a composition (A) comprising one or more reducing agents has been used, the oxidizing composition (B) as defined above is then applied, and is generally left to act for 1 minute to one hour, and then the hair is generally thoroughly rinsed, generally with water.

Final shampooing may optionally be carried out.

The step of heating the hair to a temperature ranging from 60° C. to 220° C. is performed as indicated previously.

Another subject of the invention is the use of the cosmetic treatment process as described previously to obtain a smooth feel of the hair, an increase in the lightness of the hair and a control of the volume of the hair in the presence of humidity.

Another subject of the invention is a first multicompartment device or kit, comprising:
a first compartment comprising a composition (A) comprising one or more thiol or non-thiol reducing agents,
a second compartment comprising an oxidizing composition (B), and
a third compartment comprising a composition (C) containing one or more silicones having a molecular weight of less than 10 000 and that are functionalized with one or more mercapto groups as defined previously.

Another subject of the invention is a second multicompartment device or kit, comprising:
a first compartment comprising a composition (A) comprising one or more thiol or non-thiol reducing agents, and one or more silicones having a molecular weight of less than 10 000 and that are functionalized with one or more mercapto groups as defined previously, and
a second compartment comprising an oxidizing composition (B).

Another subject of the invention is a cosmetic composition comprising:
one or more silicones having a molecular weight of less than 10 000 and that are functionalized with one or more mercapto groups corresponding to the formula:

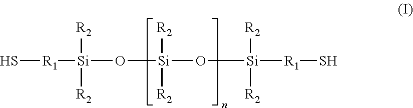

n, R₁ and R₂ having the same meanings as above,

and
one or more agents for breaking disulfide bonds of keratin.

A final subject of the invention is a cosmetic composition comprising:
one or more silicones having a molecular weight of less than 10 000 and that are functionalized with one or more mercapto groups corresponding to the formula:

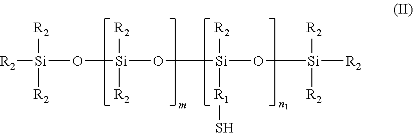

n₁, m, R₁ and R₂ having the same meanings as above,

in a content ranging from 2% to 9% by weight, relative to the total weight of the composition, and
one or more agents for breaking disulfide bonds of keratin in a content ranging from 0.5% to 3% by weight, relative to the total weight of the composition.

Preferably, the agent(s) for breaking disulfide bonds of keratin of the hair are chosen from thiol reducing agents, non-thiol reducing agents, and mineral or organic hydroxides and/or precursors thereof.

The invention is illustrated by the following examples.

### EXAMPLES

### Compositions Tested

The compositions of which the formulations are given in the table below are prepared. The amounts are given as weight percentages relative to the total weight of the composition.

The compositions of examples 1 to 4 are compositions according to the invention.

| Composition | Ex. 1 | Ex. 2 | Ex. 3 | Ex. 4 | Control 1 | Control 2 | Control 3 | Control 4 | Control 5 |
|---|---|---|---|---|---|---|---|---|---|
| 71% solution of ammonium | 11.16% | 11.16% | 7.5% | 7.5% | | 7.5% | 7.5% | 7.5% | 11.16% |
| thioglycolate in water | | | | | | | | | |
| Diethylenetriaminepentaacetic | 0.4% | 0.4% | 0.4% | 0.4% | | 0.4% | 0.4% | 0.4% | 0.4% |
| acid, pentasodium salt as | | | | | | | | | |
| a 40% aqueous solution | | | | | | | | | |
| Ammonium bicarbonate | 2% | 2% | 1.34% | 1.34% | | 1.34% | 1.34% | 1.34% | 2% |
| Silicone with chain-end | 2% | | 2% | | 2% | | | | |
| mercapto groups (M < | | | | | | | | | |
| 10000) (1) | | | | | | | | | |
| Silicone with pendent | | 2% | | 2% | | | | | |
| mercapto groups (M < | | | | | | | | | |
| 10000) (2) | | | | | | | | | |
| Silicone with pendent | | | | | | 2% | | | |
| mercapto groups (M > | | | | | | | | | |
| 10000) (3) | | | | | | | | | |
| Unfunctionalized silicone | | | | | | | 2% | | |
| (M < 10000) (4) | | | | | | | | | |
| Guar gum (5) | 1% | 1% | 1% | 1% | 1% | 1% | 1% | 1% | 1% |
| Propylene glycol | 5% | 5% | 5% | 5% | 5% | 5% | 5% | 5% | 5% |
| Fragrance | 0.3% | 0.3% | 0.3% | 0.3% | 0.3% | 0.3% | 0.3% | 0.3% | 0.3% |
| Peptizer | 0.6% | 0.6% | 0.6% | 0.6% | 0.6% | 0.6% | 0.6% | 0.6% | 0.6% |
| Ammonia | qs | qs | qs | qs | qs | qs | qs | qs | qs |
| | pH = 8.5 | pH = 8.5 | pH = 8.5 | pH = 8.5 | pH = 8.5 | pH = 8.5 | pH = 8.5 | pH = 8.5 | pH = 8.5 |
| Water | qs | qs | qs | qs | qs | qs | qs | qs | qs |

(1) sold by the company SHIN-ETSU under the commercial reference X-22-167B
(2) sold by the company SHIN-ETSU under the commercial reference KF-2001
(3) sold by the company SHIN-ETSU under the commercial reference KF-2004
(4) sold by the company SHIN-ETSU under the commercial reference KF-96 A-6CS
(5) sold by the company RHODIA under the commercial reference JAGUAR HP 105

Application Protocol

Each composition is applied to a 2.7 g brown Caucasian lock of hair according to the following protocol:
application of a Garnier Ultra-Doux Camomille shampoo to the hair,
impregnation of the lock of hair by the composition with a bath ratio of 2,
left on for 15 minutes,
rinsing of the hair,
application of an 8-volume Dulcia Vital DV2 oxidizing solution for 5 minutes,
rinsing of the hair,
predrying of the hair with a hairdryer,
application of a straightening iron at a temperature of 180° C.

Evaluation

The locks are evaluated sensorially according to the criteria of smoothness, of evenness of the fiber and of feeling of decrease of the weight.

The resistance to humidity is also evaluated (change in the volume of the lock in the presence of humidity) using the following test: the locks are placed in a glove box at 80% humidity at 20° C. for 17 hours. Images of the locks at the start time and after 17 hours at 80% humidity at 20° C. are compared.

The locks are evaluated after the application according to the protocol defined above, then after 5 shampooing operations, then after 10 shampooing operations.

### RESULTS

The locks treated with the compositions of Examples 1 to 4 have a smooth, even feel and a reduction in the feeling of hair weight, after application and after 5 shampooing operations.

Their increase in volume in the presence of humidity, after application or after 5 shampooing operations, is very low compared to an untreated control lock.

The lock treated with the composition according to Example 3 was evaluated sensorially by a panel of 7 experts compared to a lock that had not undergone any treatment. 7 experts out of 7 evaluate the treated lock as being finer, smoother and more homogeneous than a lock that had not undergone any cosmetic treatment.

A lock treated with the control composition 1, which contains no reducing agent, has a smooth feel but does not exhibit resistance to humidity.

A lock treated with the control composition 2, which contains a mercaptosiloxane having a weight greater than 10 000, exhibits resistance to humidity. On the other hand, no feeling of reduction of hair weight is experienced.

A lock treated with the control composition 3, which contains an unfunctionalized silicone, exhibits resistance to humidity. On the other hand, no feeling of reduction of hair weight is experienced.

1. A process for the cosmetic treatment of the hair during an operation for permanently reshaping the hair, comprising:
a step of breaking the disulfide bonds of keratin, by applying, to the keratin fibers, a composition (A) comprising one or more agents for breaking disulfide bonds of keratin, then optionally,
a fixing step aimed at reclosing said disulfide bonds, by applying an oxidizing composition (B) to the keratin fibers in the case of the use of one or more reducing agents as breaking agent,
it being understood that one or more silicones (i) having a molecular weight of less than 10 000 and that are functionalized with one or more mercapto groups are introduced into the composition (A) and/or into the oxidizing composition (B) and/or are applied to the keratin fibers between the step of applying the composition (A) and the step of fixing by applying the oxidizing composition (B), using an intermediate composition (C) containing it or them,
said process comprising a step of heating the hair to a temperature ranging from 60 to 220° C. after application of the silicone(s) (i).

2. The process as claimed in claim 1, characterized in that the silicone(s) (i) are chosen from compounds having the following formulae: 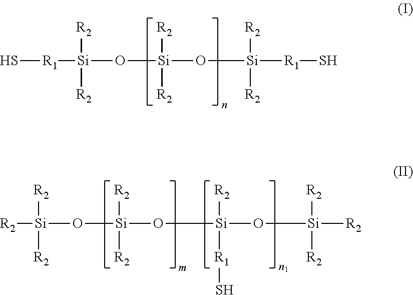
in which:
R₁ denotes a saturated or unsaturated, linear or branched, optionally cyclic hydrocarbon-based chain comprising from 1 to 100 carbon atoms, optionally interrupted with a heteroatom chosen from N, O, S and P,
R₂ denotes an alkyl group containing from 1 to 6 carbon atoms or an alkoxy group containing from 1 to 6 carbon atoms,
n ranges from 0 to 132,
n₁ ranges from 1 to 132, and
m ranges from 1 to 132.

3. The process as claimed in claim 1, characterized in that the content of functionalized silicone (i) in compositions (A) and/or (B) and/or (C) ranges from 0.5% to 50% by weight, preferably from 0.5% to 5% by weight and better still from 1% to 2% by weight relative to the total weight of the composition containing it.

4. The process as claimed in claim 1, characterized in that the composition (A) comprises one or more agents for breaking disulfide bonds of keratin chosen from thiol reducing agents, non-thiol organic reducing agents, and mineral and/or organic hydroxides and/or precursors thereof.

5. The process as claimed in claim 4, characterized in that the thiol reducing agents are chosen from thioglycolic acid, thiolactic acid, glycerol monothioglycolate, cysteamine, N-acetylcysteamine, N-propionylcysteamine, cysteine, N-acetylcysteine, thiomalic acid, pantetheine, 2,3-dimercaptosuccinic acid, N-(mercaptoalkyl)-ω-hydroxyalkylamides, N-mono- or N,N-dialkylmercapto-4-butyramides, aminomercaptoalkylamides, derivatives of N-(mercaptoalkyl)succinamic acids and of N-(mercaptoalkyl)succinimides, alkylaminomercaptoalkylamides, the azeotropic mixture of 2-hydroxypropyl thioglyconate and (2-hydroxy-1-methyl)ethyl thioglycolate, mercaptoalkylaminoamides, N-mercaptoalkylalkanediamides, and salts thereof.

6. The process as claimed in claim 4, characterized in that the non-thiol reducing agents are chosen from phosphines, sulfites, sulfinic compounds, borohydrides, reducing sugars and reductones.

7. The process as claimed in claim 4, characterized in that the mineral or organic hydroxides or precursors thereof are chosen from sodium hydroxide, lithium hydroxide, potassium hydroxide, calcium hydroxide, guanidine hydroxide and guanidine carbonate.

8. The process as claimed in claim 4, characterized in that the agent(s) for breaking disulfide bonds of keratin represent from 0.1% to 50%, preferably from 2% to 10% by weight relative to the total weight of the composition (A).

9. The process as claimed in claim 1, characterized in that the oxidizing composition (B) comprises one or more oxidizing agents chosen from hydrogen peroxide, alkali metal bromates, polythionates, persalts, such as perborates, percarbonates and persulfates, adsorbed or non-adsorbed metal salts, and enzymes of the 2-electron oxidase family, and preferably hydrogen peroxide.

10. The process as claimed in claim 1, characterized in that the step of heating the hair is performed at a temperature ranging from 80 to 220° C., preferably from 100 to 220° C. and better still from 100 to 180° C.

11. The use of the process as claimed in claim 1, to obtain a smooth feel of the hair, an increase in the lightness of the hair and a control of the volume of the hair in the presence of humidity.

12. A multicompartment device or kit comprising:
a first compartment comprising a composition (A) comprising one or more thiol or non-thiol reducing agents,
a second compartment comprising an oxidizing composition (B), and
a third compartment comprising a composition (C) containing one or more silicones (i) having a molecular weight of less than 10 000 and that are functionalized with one or more mercapto groups as defined in claim 2.

13. A multicompartment device or kit comprising a first compartment comprising:
a composition (A) comprising one or more thiol or non-thiol reducing agents, and one or more silicones (i) having a molecular weight of less than 10 000 and that are functionalized with one or more mercapto groups as defined in claim 2,
a second compartment comprising an oxidizing composition (B).

14. A cosmetic composition comprising:
one or more silicones (i) having a molecular weight of less than 10 000 and that are functionalized with one or more mercapto groups corresponding to the formula: 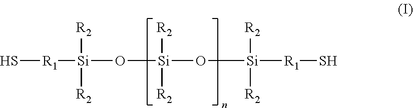
R₁ is a saturated or unsaturated, linear or branched, optionally cyclic hydrocarbon-based chain comprising from 1 to 100 carbon atoms, optionally interrupted with a heteroatom chosen from N, O, S and P,
R₂ is an alkyl group containing from 1 to 6 carbon atoms or an alkoxy group containing from 1 to 6 carbon atoms,
n varies from 0 to 132 and m varies from 1 to 132, and
one or more agents for breaking disulfide bonds of keratin of the hair.

15. A cosmetic composition comprising:
one or more silicones having a molecular weight of less than 10 000 and that are functionalized with one or more mercapto groups corresponding to the formula: 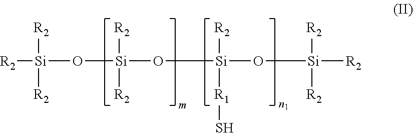
in which:
R₁ denotes a saturated or unsaturated, linear or branched, optionally cyclic hydrocarbon-based chain comprising from 1 to 100 carbon atoms, optionally interrupted with a heteroatom chosen from N, O, S and P,
R₂ denotes an alkyl group containing from 1 to 6 carbon atoms or an alkoxy group containing from 1 to 6 carbon atoms,
n₁ varies from 1 to 132 and m varies from 1 to 132,
in a content ranging from 2% to 9% by weight, relative to the total weight of the composition, and
one or more agents for breaking disulfide bonds of keratin of the hair in a content ranging from 0.5% to 3% by weight, relative to the total weight of the composition.

16. The composition as claimed in claim 14, characterized in that the agent(s) for breaking disulfide bonds of keratin of the hair are chosen from thiol reducing agents, non-thiol reducing agents, and mineral and/or organic hydroxides and/or precursors thereof.

17. The composition as claimed in claim 15, characterized in that the agent(s) for breaking disulfide bonds of keratin of the hair are chosen from thiol reducing agents, non-thiol reducing agents, and mineral and/or organic hydroxides and/or precursors thereof.
